(19) United States
(12) Patent Application Publication
(10) Pub. No.: US 2004/0185092 A1
(43) Pub. Date: Sep. 23, 2004

(54) Pharmaceutical preparations of omeprazole and/or clarithromycin for oral use

(76) Inventors: Yih Ming Hsiao, New York, NY (US); Grace C. Hsiao, Berkeley, CA (US)

Correspondence Address: FRISHAUF, HOLTZ, GOODMAN & CHICK, PC, 767 THIRD AVENUE, 25TH FLOOR, NEW YORK, NY, 10017-2023, US

(21) Appl. No.: 10/390,856

(22) Filed: Mar. 18, 2003

(51) Int. Cl. A61K 9/48

(52) U.S. Cl. 424/451; 514/37

(57) ABSTRACT

The present invention relates to a pharmaceutical preparation for oral use which is in the form of enteric coated hard or soft capsule containing omeprazole or a pharmaceutically acceptable salt thereof and/or clarithromycin or an ester thereof. This preparation is resistant to dissolving in acidic media and easy to dissolve in neutral to alkaline media. This preparation is useful in the treatment of gastrointestinal diseases.

The present invention related to a new stable and absorbable pharmaceutical preparation containing omeprazole or a pharmaceutically acceptable salt thereof and/or clarithromycin or an ester thereof for oral use, to a method for the manufacture of such a preparation, and to a method of affecting gastric acid secretion and/or Helicobacter pylori eradication rates and providing gastrointestinal cytoprotective effect when using the preparation.

### BACKGROUND OF THE INVENTION

Omeprazole, 5-methoxy-2-(((4-methoxy-3,5-dimethyl-2-pyridinyl)methyl)sulfinyl) 1H-benzimidazole, is an inhibitor of the gastric acid secretion (U.S. Pat. No. 4,255,431). It can be used for the treatment of gastric and duodenal ulcers. The stability of omeprazole is a function of pH. Omeprazole is rapidly degraded in acidic media, but has acceptable stability under alkaline conditions.

When omeprazole is administered orally in solid dosage form as a tablet, capsule or suspension, the rate of absorption is controlled by how fast the drug dissolves in the fluids at the absorption site. In other words, the rate of absorption in the oral process depends on the dissolution rate. Omeprazole is rapidly degraded in the gastric fluid, but it is stable in the duodenal and intestinal fluids. Therefore, in U.S. Pat. No. 4,786,505, the cores, i.e., pellets or tablets of omeprazole, are coated by the enteric coating agents.

Clarithromycin, 6-O-methylerythromycin, is a semi-synthetic macrolide antibiotic. Clarithromycin exerts its antibacterial action by binding to the 50S ribosomal subunit of susceptible microorganisms thereby resulting in inhibition of protein synthesis. It has demonstrated in vitro activity against Helicobacter pylori isolated from patients with duodenal ulcers. The antibiotic is destroyed by gastric acid. It is acceptably stable in the weak acidic to alkaline conditions.

It has been conventional in the prior art that in order to treat gastric and duodenal ulcers clarithromycin and omeprazole can be orally administered at the same time or in sequence. However, a capsule containing both clarithromycin and omeprazole as the active ingredients has never been disclosed or used in the prior art.

### OUTLINE OF THE INVENTION

The object of the present invention is to provide a pharmaceutical preparation for oral use which is in the form of enteric coated hard or soft capsule containing omeprazole or a pharmaceutically acceptable salt thereof and/or clarithromycin or an ester thereof. The enteric coating on the surface of the capsule may be one which is resistant to release of the active compound(s) for a period of 120 minutes at the gastric fluids whereby the active compound(s) is(are) released in the duodenal and/or intestinal fluids. This preparation is resistant to dissolving in acidic media and readily dissolves in neutral to alkaline media. This preparation is useful in the treatment of gastrointestinal diseases, and it also has a good stability during long-term storage.

### DETAILED DESCRIPTIONS OF THE INVENTION

(1) Capsules Containing Omeprazole

Omeprazole has a suitable crystal size for rapid dissolution in water or intestinal fluid. It is mixed with inert pharmaceutical excipients, and if necessary, made into granules by using wet methods, and then optionally mixed with lubricants. The resultant solid mixture is packed into empty capsules. The capsule may be a hard or soft gelatin capsule. The excipients may be one or more members selected from the group consisting of lactose, mannitol, microcrystalline cellulose, disodium phosphate, hydroxypropyl cellulose, sodium lauryl sulfate, talc and silicon dioxide, . . . etc.

(2) Capsules Containing Clarithromycin

Clarithromycin has a suitable crystal size for rapid dissolution in water or intestinal fluid. It is mixed with inert pharmaceutical excipients, and if necessary, made into granules by using wet methods, and then optionally mixed with lubricants. The resultant solid mixture is packed into empty capsules. The capsule may be a hard or soft gelatin capsule. The excipients may be one or more members selected from the group consisting of povidone, croscarmellose sodium, microcrystalline cellulose, talc and magnesium stearate, . . . etc.

(3) Capsules Containing Clarithromycin and Omeprazole

Clarithromycin and omeprazole both have a suitable crystal size for rapid dissolution in water or intestinal fluid. They are mixed with inert pharmaceutical excipients, and if necessary, made into granules by using wet granulation methods, and then optionally mixed with lubricants. The resultant solid mixture is packed into empty capsules. The capsule may be a hard or soft gelatin capsule. The excipients may be one or more members selected from the group consisting of povidone, croscarmellose sodium, microcrystalline cellulose, talc and magnesium stearate, . . . etc.

(4) Enteric coatings for pills and compressed tablets have been used for more than one century, but they have never been used for hard or soft capsules. In the present invention, an enteric coating is applied on a hard or soft capsule which contains omeprazole or a pharmaceutically acceptable salt thereof and clarithromycin or an ester thereof. An ideal enteric coating material has the following properties:

1. good resistance to gastric fluids,

2. rapid dissolution in intestinal fluids,

3. good compatibility with most drug substances and pharmaceutical excipients, and

4. good capability to form continuous films.

Suitable enteric coating materials include cellulose acetate phthalate, hydroxypropyl methylcellulose acetate succcinate, methylmethacrylate-methacrylic acid copolymer, cellulose acetate succinate, shellac, polyvinyl acetate phthalate, polyvinyl butyrate phthalate and hydroxypropyl methylcellulose phthalate, . . . etc.

The enteric coating materials may contain one or more components selected from polyethylene glycol, triethyl citrate, polyvinyl-pyrrolidone, polyvinyl alcohol, hydroxyproopyl methylcellulose, talc, purified water and alcohol, . . . etc.

### EXAMPLES

(1) The following I, II and II are examples of the formulae of omeprazole-containing capsules:

TABLE 2

Amount, mg

| Ingredient | I | II | III |
|---|---|---|---|
| Omeprazole | 10.0 | 20.0 | 40.0 |
| Lactose | 70.0 | 60.0 | 40.0 |
| Mannitol | 20.0 | 20.0 | 20.0 |
| Hydroxypropyl Cellulose | 0.5 | 0.5 | 0.5 |
| Disodium Hydrogen Phosphate | 0.5 | 0.5 | 0.5 |
| Sodium Lauryl Sulfate | 0.5 | 1.0 | 2.0 |
| Talc | 0.5 | 0.5 | 0.5 |
| Total | 102.0 | 102.5 | 103.5 |

The ingredients in each formula are mixed well, made into granules, dried, passed through mesh #30 and packed into capsules having suitable sizes.

(2) The following I, II and III are examples of the formulae of clarithromycin-containing capsules:

TABLE 1

| | Amount, mg | | |
|---|---|---|---|
| Ingredient | I | II | III |
| Clarithromycin | 125.0 | 250.0 | 500.0 |
| Povidone | 19.0 | 38.0 | 75.0 |
| Croscarmellose Sodium | 10.0 | 20.0 | 40.0 |
| Microcrystalline Cellulose | 50.0 | 100.0 | 150.0 |
| Talc | 1.0 | 2.0 | 4.0 |
| Magnesium Stearate | 1.0 | 2.0 | 4.0 |
| Total | 206.0 | 412.0 | 773.0 |

The ingredients in each formula are mixed well, made into granules, dried, passed through mesh #30 and packed into capsules having suitable sizes.

(3) The following I, II and III are examples of the formulae of clarithromycin- and omeprazole-containing capsules:

TABLE 3

| | Amount, mg | | |
|---|---|---|---|
| Ingredient | I | II | III |
| Omeprazole | 10.0 | 20.0 | 10.0 |
| Clarithromycin | 125.0 | 125.0 | 250.0 |
| Microcrystalline Cellulose | 100.0 | 100.0 | 100.0 |
| Povidone | 35.0 | 40.0 | 40.0 |
| Talc | 1.5 | 2.0 | 2.0 |
| Magnesium Stearate | 1.5 | 2.0 | 4.0 |
| Total | 262.0 | 289.0 | 404.0 |

The ingredients in each formula are mixed well, made into granules, dried, passed through mesh #30 and packed into capsules having suitable sizes.

(4) The followings are examples of formulae of enteric coating solutions for omeprazole and/or clarithromycin capsules:

TABLE 4

| | Amount, percent (%) | | | | |
|---|---|---|---|---|---|
| Ingredient | I | II | III | IV | V |
| Methacrylic acid | 8.0 | — | — | — | — |
| copolymer | | | | | |
| Hydroxypropyl | — | 8.5 | — | — | — |
| methylcellulose | | | | | |
| Phthalate | | | | | |
| Cellulose acetate | — | — | 7.5 | — | — |
| Phthalate | | | | | |
| Shellac | — | — | — | 8.0 | — |
| Polyvinyl acetate | — | — | — | — | 8.0 |
| phthalate | | | | | |
| Triethyl citrate | 1.2 | — | — | — | — |
| Polyethylene glycol | — | 1.4 | — | — | — |
| Propylene glycol | — | — | 1.3 | — | — |
| Castor oil | — | — | — | 1.0 | — |
| Glycerin | — | — | — | — | — |
| Talc | 3.0 | 3.0 | 3.0 | — | 3.0 |
| Purified water q.s. | 100.0 | 100.0 | 100.0 | — | 100.0 |
| Alcohol q.s. | — | — | — | 100.0 | — |

The ingredients in each formula except formula IV are mixed well and homogenized. The ingredients in formula IV should be mixed until the solids are dissolved.

(5) Method of producing the pharmaceutical preparation of the present invention:

The capsules which contain clarithromycin and/or omeprazole are placed into a coating pan or the Glatt coater. They are coated with the enteric coating solution until the sufficient amount of enteric coating materials cover the surface of the capsules, which do not break down in the gastric fluids and can release omeprazole and clarithromycin in the intestinal fluids. Each coated capsule thus obtained may contain 50˜1000 mg of clarithromycin, and may contain 5˜30 mg of omeprazole.

### Experiments

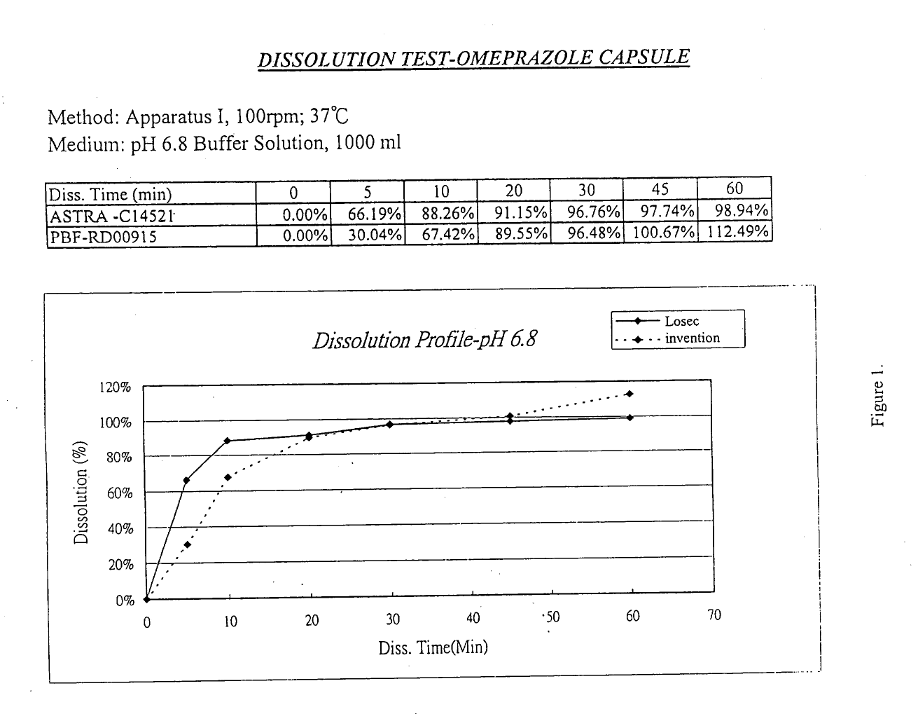

(1) The enteric coated capsules containing omeprazole produced according to the present invention, and Losec® 20-mg capsules which are conventional omeprazole-containing capsules commercially available from AstraZeneca AB, Sweden, are compared with respect to their dissolution rates. The results are shown in Table 5, and the dissolution profiles are shown in FIG. 1.

TABLE 5
Method: using Apparatus I, 100 rpm; 37° C.
Medium: pH 6.8 Buffer Solution, 1000 ml

| | Dissolution Time (min) | | | | | | |
|---|---|---|---|---|---|---|---|
| | 0 | 5 | 10 | 20 | 30 | 45 | 60 |
| Losec | 0.00% | 66.19% | 88.26% | 91.15% | 96.76% | 97.74% | 98.94% |
| invention | 0.00% | 30.04% | 67.42% | 89.55% | 96.48% | 100.67% | 112.49% |

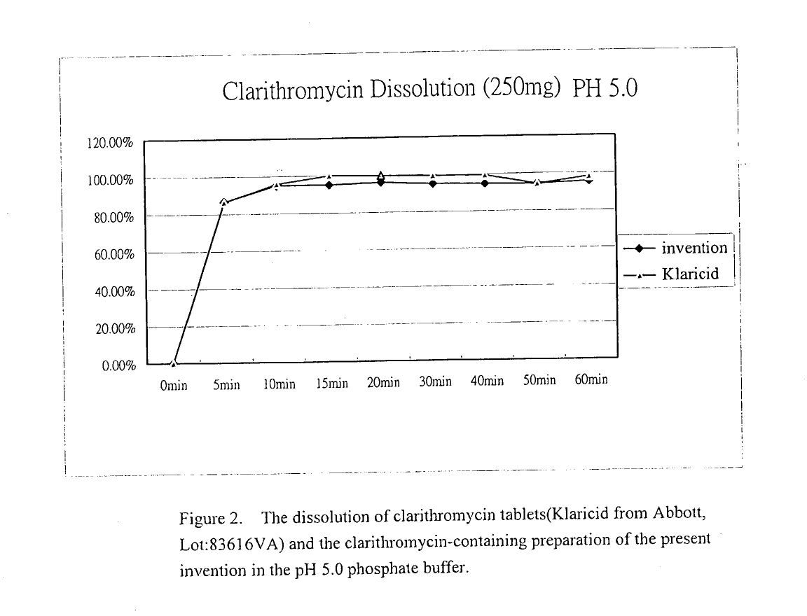

(2) The enteric coated capsules containing omeprazole produced according to the present invention, and Klaricid® tablets, Lot: 83616VA, which are conventional clarithromycin-containing tablets commercially available from Abbott, are compared with respect to their dissolution rates. The dissolution profiles are shown in FIG. 2.

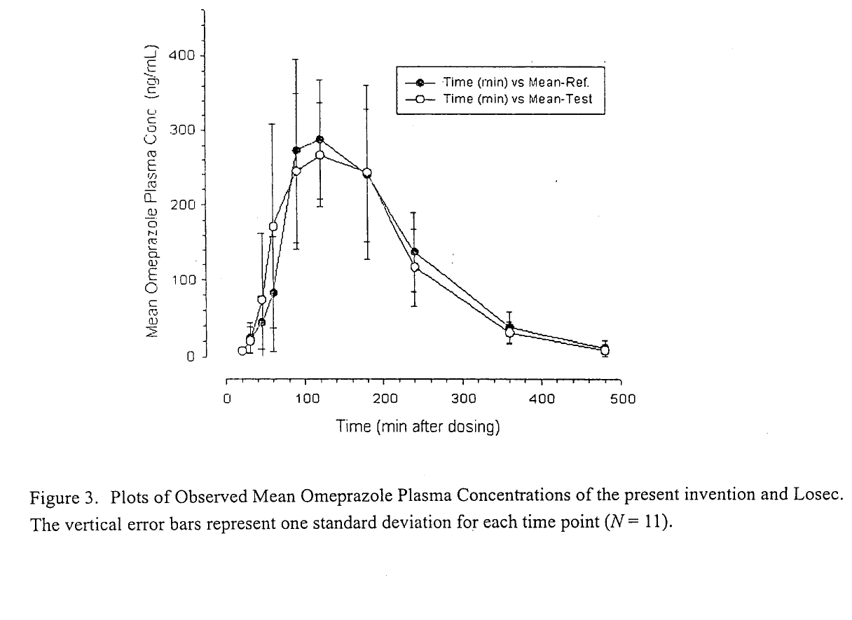

(3) Clinical tests are conducted on eleven healthy male adults (age: 26-35 years old, average 29 years old; body weight: 65-80 kg, average 71 kg), by using the enteric coated capsules containing omeprazole produced according to the present invention, and Losec® 20-mg capsules which are conventional omeprazole-containing capsules commercially available from AstraZeneca AB, Sweden. The mean omeprazole plasma concentration-time curve after oral administration with a single dose of 20 mg of either omeprazole products in the 11 normal male volunteers are shown in FIG. 3. and the dissolution profiles are shown in FIG. 1. The statistical summaries for this test are shown in Table 6.

TABLE 6

| | Losec | | Invention | | |
|---|---|---|---|---|---|
| | | (Standard | | (Standard | Mean Ratio |
| Parameters | Mean | Deviation) | Mean | Deviation) | Invention/Losec |
| Cₘₐₓ (ng/ml) | 347 | (73) | 347 | (58) | 1.03 |
| AUC₀₋ₗₐₛₜ(ng * min/mL) | 56661 | (13971) | 58284 | (9798) | 1.01 |
| AUC_{0-inf}(ng * min/mL) | 57464 | (14042) | 55170 | (10066) | 1.02 |
| ln(Cₘₐₓ) | 5.83 | (0.22) | 5.84 | (0.17) | 1.00 |
| ln(AUC₀₋ₗₐₛₜ) | 10.9 | (0.3) | 10.9 | (0.2) | 1.00 |
| ln(AUC_{0-inf}) | 10.9 | (0.3) | 10.9 | (0.2) | 1.00 |

wherein
Cₘₐₓ is the mean omeprazole peak plasma concentration;
AUC₀₋ₗₐₛₜ is the area under the curve calculated from time zero to the time of the last measurable omeprazole concentration; and
AUC_{0-inf} is the area under the curve calculated from time zero to the time infinite.

### The Advantages of the Present Invention

A. The method of the present invention for producing the special pharmaceutical preparations of the present invention is easier to operate and can reduce the cost, as compared with the conventional methods of producing enteric coated pellets or tablets.

B. The method of the present invention for producing the special pharmaceutical preparations of the present invention is easier to operate and can reduce the cost, as compared with the conventional method disclosed in U.S. Pat. No. 4,786,505 in which two subcoat layers are formed on the surface of the omeprazole pellets.

C. The pharmaceutical preparations of the present invention have lower dissolution rates in acidic media when compared with conventional products containing omeprazole or clarithromycin, as can be seen from FIGS. 1 and 2. Therefore they are not rapidly disintegrated in the gastric fluids.

D. The pharmaceutical preparations of the present invention are not rapidly disintegrated in the gastric fluids, therefore when they are used in the treatment of gastric and duodenal ulcers have a better release-controlled property than that of the conventional products.

E. It is seen from the data in Table 6 that there are no significant differences between the pharmaceutical preparations of the present invention and Losec®, a conventional omeprazole capsule, in their pharmacokinetic bioequivalence. However, the products of the present invention are better than Losec® since the former have smaller standard deviations of the Cₘₐₓ, AUC₀₋ₗₐₛₜ and AUC_{0-inf} data than the latter does.

We claim:

1. A pharmaceutical preparation for oral use comprising a coated capsule which contains substance(s) selected from one of the following items (1)˜(3):
(1) as an active compound, omeprazole or a pharmaceutically acceptable salt thereof,
(2) as an active compound, clarithromycin or an ester thereof,
(3) as both active compounds, omeprazole or a pharmaceutically acceptable salt thereof, and clarithromycin or an ester thereof,
wherein the coating on the capsule comprises one or more enteric coating materials whereby the active compound(s) is(are) released in the intestine.

2. A pharmaceutical preparation as claimed in claim 1, wherein the coating is resistant to release of the active compound(s) for a period of 120 minutes at the gastric fluids whereby the active compound(s) is(are) released in the duodenal and/or intestinal fluids.

3. A pharmaceutical preparation as claimed in claim 1, wherein the enteric coating material is selected from shellac, methacrylic acid copolymer, cellulose acetate phthalate, hydroxypropyl methylcellulose phthalate, polyvinyl acetate phthalate, ethylcellulose, polyvinyl acetate, and poly(ethylacrylate-methylmethacrylate), . . . etc.

4. A pharmaceutical preparation as claimed in claim 1, wherein the enteric coating material optionally contains a plasticizer.

5. A pharmaceutical preparation as claimed in claim 1, which is useful in the treatment of gastrointestinal diseases.

6. A pharmaceutical preparation as claimed in claim 1, which comprises omeprazole in a unit dose of 5 to 30 mg, and/or clarithromycin in a unit dose of 50 to 1000 mg.

7. A pharmaceutical preparation as claimed in claim 1, wherein the capsule is a hard or soft gelatin capsule.
